(19) United States
(12) Patent Application Publication
(10) Pub. No.: US 2005/0250099 A1
(43) Pub. Date: Nov. 10, 2005

(54) Direct pcr quantification

(75) Inventor: Stuart Hassard, London (GB)

Correspondence Address: WALLENSTEIN WAGNER & ROCKEY, LTD, 311 SOUTH WACKER DRIVE, 53RD FLOOR, CHICAGO, IL, 60606, US

(73) Assignee: Deltadot Limited, London (GB)

(21) Appl. No.: 10/515,934

(22) PCT Filed: Jun. 2, 2003
(86) PCT No.: PCT/GB03/02390

§ 371 Date: Jun. 13, 2005

(30) Foreign Application Priority Data
May 31, 2002 (GB) 0212764.5

(51) Int. Cl. C12Q 1/68; C12P 19/34; C12M 1/34

(52) U.S. Cl. 435/6; 435/91.2; 435/287.2

(57) ABSTRACT

An apparatus and method for analysing temperature-dependent molecular configurations such as folding comprises a multi-channel flow-through chip (12) along which molecules to be analysed pass. A temperature gradient is maintained along the length of the chip. As molecules pass along the channels they fold or unfold, in response to the changing temperature. These changing molecular configurations are investigated by simultaneously measuring the extent to which the molecules absorb UV light, and the extent to which they fluoresce. The absorption and fluorescence information is supplied to a computer system (26) for real-time analysis.

The present invention relates to a process to monitor the production of coherent nucleic acid molecules from an inherent PCR mixture; and to an apparatus for such a process.

PCR is an elegant technique to increase the amount of a particularly interesting piece of nucleic acid. This may be a gene or part of a molecular control mechanism, which needs to be sequenced or analysed by restriction enzyme digestion. Whatever it is, having a plentiful supply is advantageous.

Not only is PCR generally useful, but real-time quantitative PCR is a powerful tool that can be used for a multitude of gene investigations, such as gene expression analysis, genotyping, pathogen detection/quantitation, mutation screening, nucleic acid quantitation and single nucleotide polymorphism (SNP) validation.

Real-time PCR quantification has already been achieved using the TaqMan® system. The TaqMan® probe (20-30 bp), disabled from extension at the 3′ end, consists of a site-specific sequence labelled with a fluorescent reporter dye and a fluorescent quencher dye. During PCR the TaqMan® probe hybridises to its complementary single strand DNA sequence within the PCR target. When amplification occurs, the TaqMan® probe is degraded due to the 5′→3′ exonuclease activity of Taq DNA polymerase, thereby separating the quencher from the reporter during extension. Due to the release of the quenching effect on the reporter, the fluorescence intensity of the reporter dye increases. During the entire amplification process this light emission increases exponentially, the final level being measured by spectrophotometry after termination of the PCR. Because increase of the fluorescence intensity of the reporter dye is only achieved when probe hybridisation and amplification of the target sequence has occurred, the TaqMan® assay offers a sensitive method to determine the presence or absence of specific sequences.

The use of probes in PCR is described in a number of papers, including: Holland PM, Abramson RD, Watson R & Gelfland DH (1991. Detection of specific polymerase chain reaction product by utilising the 5′→3′ exonuclease activity of Thermus aquaticus DNA polymerase, and Proceedings of the National Academy of Sciences USA, 88: 7276-7280. Lee L G, Connell C R & Block W (1993). Allelic discrimination by nick-translation PCR with fluorogenic probes. Nucleic Acid Research, 21: 3761-3766.

However, the TaqMan® system is associated with various disadvantages, including the following:
it is complex and expensive
an expensive primer system is required
PCR is a very optimised system and the addition of unnecessary components can cause a loss of efficiency
contaminated PCR product.

It would therefore be of great benefit if a system for the direct quantification of PCR product could be created, which overcame or alleviates one or more of the above problems. The present invention provides such a system.

The first aspect of the invention provides a process to monitor the production of coherent nucleic acid molecules from an inherent PCR mixture, the process comprising collating PCR starting materials in a chamber, and carrying out the PCR steps of denaturation, annealing and extension, while monitoring the production of coherent nucleic acid molecules by shining a UV light into the mixture and determining the amount of light absorbed by the said molecules.

Collating the PCR starting materials is simply a case of assembling or collecting the starting materials. The PCR starting materials, which usually comprise an incoherent PCR mixture are subjected to the usual PCR steps to enable, the production of coherent nucleic acid molecules. The molecular events are monitored in real time by shining a UV light into the mixture and determining the extent to which that light is absorbed by the intrinsic absorption capabilities of the nucleic acid molecules. Preferably, the UV light is shone through the mixture and the amount of light passing through is monitored. By determining how the amount of transmitted light varies with time, the amount of absorption and hence the quantity of nucleic acid molecules can be determined. The monitoring can thus be done in real time.

The benefits of such a process and system include:
No outlay on expensive primer systems,
Real-time measurement, and
Recoverable, uncontaminated PCR product.

The PCR in the present invention includes all nucleic acid amplification systems which include the steps of denaturation, annealing and extension (although not necessarily in that order). Such systems are well known in the art and the basic PCR uses four main components, as follows:
1. The nucleic acid fragment (usually DNA) which contains the sequence to be amplified—the target sequence. Theoretically, only a single piece of this nucleic acid is needed. Usually a double stranded piece is used.
2. The primers that flank the target sequence. A primer is a small piece of nucleic acid (again, usually DNA) that complements the sequence flanking the target. In order for a new strand of nucleic acid to be created it must have a foundation to be made from. That is the function of the primer. Primers are always added in excess—more is added to the reaction than could possibly be used.
3. A free solution of dinucleotide triphosphates (dNTPs)—the base part of a base-pair. The new strands of nucleic acid are made from these dNTPs, A-T, C-G. As with the primers, these are present in excess.
4. The thermostable polymerase (usually DNA polymerase). This is the enzyme, such as Taq polymerase or any other thermostable DNA polymerase, that mediates the whole reaction.

PCR is achieved in three distinct steps, Denaturation, Annealing and Extension. One set of these three phases is called a Cycle. A typical PCR consists of 30 such cycles.

Denaturation

The target nucleic acid in its natural double-stranded form is inaccessible to the primers because they can only stick (or anneal) to single stranded nucleic acid. Incubation at temperatures in the region of 95° C. for a short time leads to denaturation of the double strand.

Annealing

The reaction is now rapidly cooled to 55° C. and held there for about 1 minute. At this temperature the primers are able to stick to the single stranded nucleic acid, but only at the specific place dictated by their sequences.

Extension

The primers are now stuck to the target sequences and synthesis of the new strands of nucleic acid can begin. This occurs in two phases. The reaction is heated to 72° C.—the temperature at which the nucleic acid polymerase becomes most active. The polymerase mediates the creation of the new nucleic acid strand, using the target nucleic acid as a template. It adds the complementary dNTP to the growing strand as it moves along the template. In the extension step the nucleic acid strands are extended from each primer to create a number of copies of the target nucleic acid that are longer than the sequence defined by the two primers. This is because there is nothing to stop the polymerase as it moves down the target nucleic acid strand. It continues making the new nucleic acid strand as long as the conditions are favourable. Of course, this is not what is wanted because it doesn't give us our PCR product.

This problem however, is only temporary, because now the reaction heats up to the 95° C. denaturation step. The polymerase stops extending the new nucleic acid strand and the two nucleic acid strands (target and new) separate. This allows the next set of primers access to them.

In the following annealing step, the primers stick to both the original and the new nucleic strand. This is only as long as the product we want so that is what is made.

PCR can be used to detect the presence of a DNA or even RNA species in a cell system because if you use the correct primers, they can pick up a gene or whatever else is sought.

Preferably, the target nucleic acid of the PCR is double stranded DNA.

Any target nucleic acid is suitable, for example human, bacterial, viral, other microbial, plant, or nucleic acid of unknown origin.

The PCR may be any which involves the steps of denaturation, annealing and extension and includes the basic PCR as described in Holland et al (Supra) or Lee et al (Supra), as well as known or future PCR such as Reverse-Transciptase PCR (RT-PCR) (Vanden Heuvel, J. P., Tyson, F. L. and Bell, D. A. Constructions of recombinant RNA templates for use as internal standards in quantitative RT-PCR, Biotechniques 14:395-395 (1994)), Long PCR (Cheng, S., Fockler, C., Barnes, W., Higuchi, R. Effective amplification of long targets from cloned inserts and human genomic DNA. Proc: Natl. Acad. Sci. 91, 5695-5699 (1994)), Hot-start PCR, “Touch-down” PCR, Inverse PCR, AP-PCR (arbitrary primed/RAPD (radon amplified polymorphic DNA)), quantitative RT-PCR, RT in situ PCR, Nested RT-PCR, RACE (rapid amplification of cDNA ends), DD-PCR (differential display), multiplex-PCR, asymmetric PCR, 3′ mismatch SNP validation and the like. The PCR may be competitive and/or quantitative.

All of the above are standard techniques and further details can be found from supplies such as Alkami Biosystem, Fermentas, Promega, Roche, Qiagen and Sigma.

The chamber for the PCR reaction may be any through which the intrinsic UV absorption of the nucleic acid can be measured. Such chambers include any UV transparent material, such as quartz, fused silica or Poly Dimethyl Siloxane (PDMS).

The chamber may be a conventional PCR vessel or may be an alternative arrangement, such as a chip as described in Kopp, M. U., de Mello, A. J., and Manz, A. Chemical Amplification: Continuous-Flow PCR on a Chip. Science, Vol 280, 1998.

In order to detect nucleic acid molecules by their intrinsic absorption of UV light, a molecular imaging device may be used. Such a device comprises a UV light source arranged to shine onto the sample to be investigated and a UV detector arranged to detect the position of molecules. A Photo Diode Array or Charge Coupled Device (CCD) can be used as a suitable detector.

Label-free intrinsic imaging may be used, as described in WO 96/35946, full content of which is incorporated by reference.

The molecules may be imaged directly onto any suitable detector, such as a diamond detector. The light source may be any suitable source, such as constant brightness UV light from either a broad spectrum device like a Helium discharge tube, a deuterium lamp or a Xenon lamp. The different amounts of light reaching the detector placed by the object being imaged is observed.

Since it is possible to measure the UV absorption of the molecules as the PCR progresses, a graph of the coherent nucleic acid molecules, real-time, can be generated. This provides information, on a real-time basis on the following:
whether any coherent nucleic acid molecules are being produced
how much
how quickly
what size

In respect of the latter bullet point (what size), the gradient of the slope will be proportional to the size of the product as the rate of production in the PCR is dependent on the enzyme used (e.g. at 72° C. Taq polymerase—60-150′, Tth polymerase—25′).

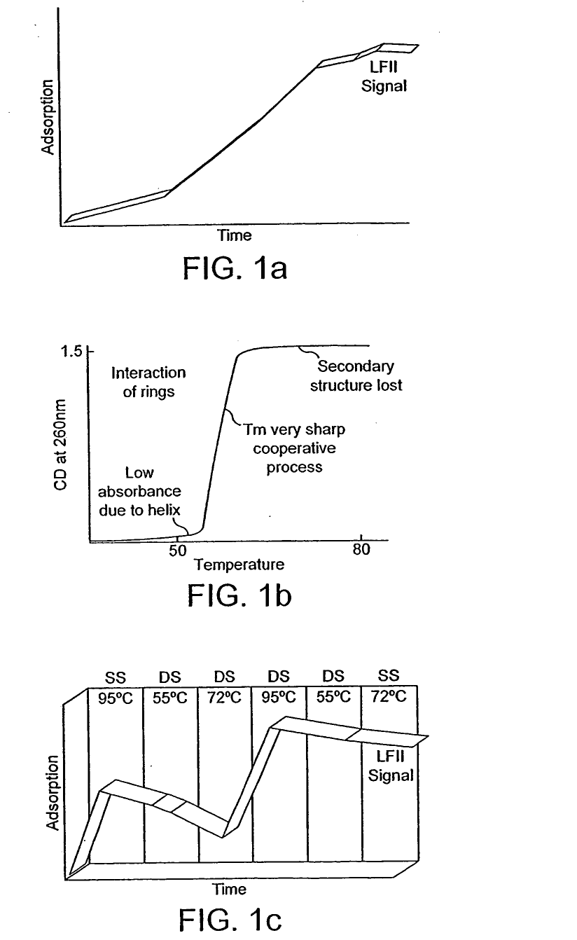

As single stranded nucleic acid molecules absorb more light than double stranded nucleic acid molecules, the curve produced by a real-time plot of absorption versus time will not be smooth; rather an image as shown in FIG. 1a and FIG. 1b.

In a second aspect, the invention provides apparatus for monitoring the production of coherent nucleic acid molecules from an incoherent PCR mixture, the apparatus comprising:
a chamber adapted for a PCR;
a UV light source adapted to shine on said chamber; and
means to detect intrinsic UV absorption of said UV light in real time.

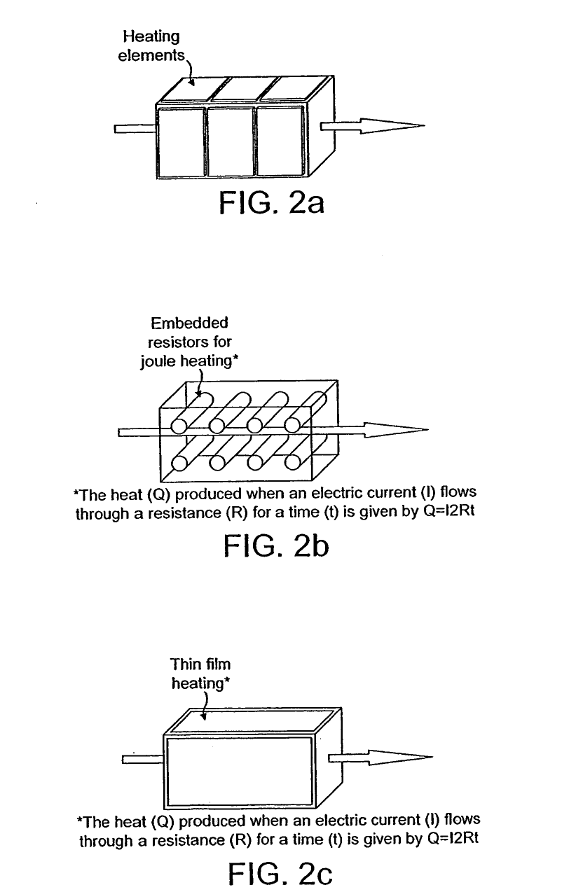

The chamber may be any as described above for the first aspect of the invention. The chamber must be suitable for PCR, i.e. allow addition of the incoherent PCR mixture as well as provide suitable means for the required thermal cycling. Suitable heating strategies for a direct PCR chamber are shown in FIG. 2(a, b and c).

The UV light source and detection means may be any as described according to the first aspect of the invention.

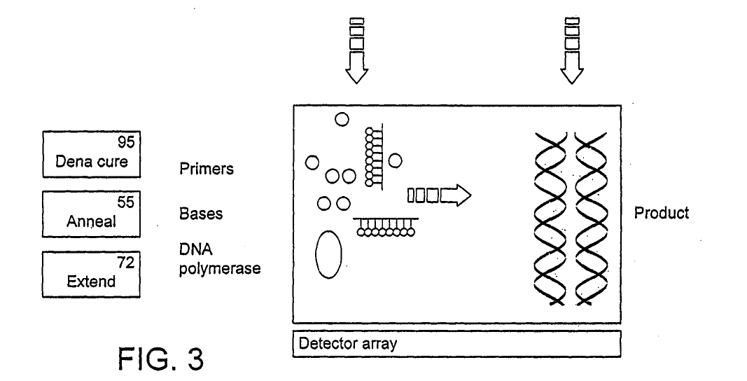

A diagrammatic representation of an apparatus can be seen in FIG. 3.

A third aspect of the invention provides use of label free intrinsic imaging to monitor the production of coherent nucleic acid molecules from an incoherent PCR mixture.

All preferred features of the first and second aspects of the invention also apply to the third.

The present invention allows an improved and real-time monitor of PCR. An advantage of the label free nucleic acid is that after the PCR, the nucleic acid can be further utilised. For example, the nucleic acid can be extracted and optionally purified. The coherent nucleic acid molecules can be separated from any remaining incoherent PCR mixture by electrophoresis, optionally with further purification. Such nucleic acid can subsequently be used in further nucleic acid manipulations, such as insertion into vectors, sequencing etc.

The present invention can also be used as a process which is a detection system to determine whether a coherent nucleic acid molecule has been produced from a PCR. Such a system does not require real-time monitoring but, rather, monitoring at some point subsequent to the start of PCR (during or at the end of the PCR). All aspects of the invention apply.

Such an end-product monitor can be used, for example, in SNP (Single Nucleotide Polymorphism) analysis (including 3′ mismatch SNP analysis). In such an embodiment of the present invention two or more chambers are provided, optionally in a side by side, or tray arrangement.

One of the chambers may contain nucleic acid representing the WT (Wild Type) allele in question and one or more additional chambers may contain a sample allele. The sample allele (or alleles) are provided for SNP analysis.

Each chamber is provided with an incoherent PCR mixture, with suitable primers allowing for amplification of the WT allele.

The chamber containing the WT allele is effectively the control. Successful amplification of the WT allele should occur wen the chamber undergoes denaturation, annealing and extension. Successful amplification of the one or more sample alleles will depend on the presence or absence of a SNP in the primer region. The monitoring of the PCR (real-time or other) will enable determination of the presence or absence of a SNP in the sample allele. Apparatus and system for such determination can be provided as a multiplex system.

The invention is described with reference to the following figures:

FIGS. 1a and 1b are graphs showing absorption versus time obtained by a method according to the first aspect of the invention. The Label Free Intrinic Imaging (LFII) signal is shown.

As the double stranded DNA product is created, the absorption will increase. In the linear phase, the slope will be a function of the rate of incorporation of nucleotides by the thermostable DNA polymerase and the length of the template/product.

FIG. 1b is also a graph showing absorption versus time. This graph being more detailed. The Hyperchromic effect increases the adsorption of UV light. This is due to the increased exposure of the optical active bases. Directly observing the PCR by LFII may allow us to see this in real-time as the temperature cycles. Theoretically, there may be an increase in signal in the 95° C. phase and a slight fall as the DNA re-natures. This will produce a stepped signal as depicted in the graph.

FIG. 2(a, b and c) shows suitable heating strategies for a direct PCR chamber.

FIG. 3 shows a diagrammatic representation of an apparatus according to the second aspect of the invention.

1. A process to monitor the production of coherent nucleic acid molecules from an inherent PCR mixture, the process comprising the steps of: collating PCR starting materials in a chamber, and carrying out the PCR steps of denaturation, annealing and extension, while monitoring the production of coherent nucleic acid molecules by shining a UV light into the mixture and determining the amount of light absorbed by the molecules.

2. A process as claimed in claim 1, wherein the incoherent PCR mixture comprises a target nucleic acid molecule which is double stranded DNA.

3. A process as claimed in claim 1, wherein the PCR is RT-PCR, Long PCR or 3′ mismatch PCR.

4. A process as claimed claim 1, wherein the chamber is of UV transparent material selected from the group consisting of material such as quartz, fused silica, and PDMS.

5. A process as claimed in claim 1 , wherein the process is monitored in real-time.

6. An apparatus for monitoring the production of coherent nucleic acid molecules from an incoherent PCR mixture, the apparatus comprising:
a chamber adapted for a PCR;
a UV light source adapted to shine on said chamber; and
means to detect intrinsic absorption of the UV light by the molecules.

7. A process to monitor the production of coherent nucleic acid molecules from an incoherent PCR mixture, the process comprising the step of:
using label free intrinsic imaging to monitor.

8. The process as claimed in claim 7, wherein the monitoring is real-time.

9. The process as claimed in claim 7, wherein such use is in SNP analysis.

10. (canceled)
